(19) United States
(12) Patent Application Publication
(10) Pub. No.: US 2016/0217707 A1
(43) Pub. Date: Jul. 28, 2016

(54) A TRAINING INSERTER

(71) Applicant: BAYER OY, Turku (FI)

(72) Inventors: Ilkka JUTILA, Sauvo (FI); Sanna VILLILÄ, Sauvo (FI)

(73) Assignee: Bayer Oy, Turku (FI)

(21) Appl. No.: 14/917,128

(22) PCT Filed: Sep. 11, 2014
(86) PCT No.: PCT/EP2014/069357

§ 371 Date: Mar. 7, 2016

(30) Foreign Application Priority Data
Sep. 11, 2013 (EP) 13183870.8

(51) Int. Cl. G09B 23/28; A61F 6/18

(52) U.S. Cl. CPC G09B 23/28; A61F 6/18

(57) ABSTRACT

The invention relates to a training inserter (1) for an intrauterine system, said intrauterine system comprising a body (11) and removal strings (17), the training inserter comprising a handle (2) having a first end (3) and a second end (4), an insertion tube (5) having a first end (6) and a second end (7), and being arranged in connection with the handle (2), for receiving the intrauterine system, a plunger arranged inside the insertion tube (5), and first means for moving the insertion tube (5) with respect to the plunger for pushing out the intrauterine system. The training inserter further comprises second means (14, 18) that perform the following functions: attaching the removal strings to the inserter, pulling back the intrauterine system via the removal strings, and storing the removal strings.

### FIELD OF THE INVENTION

The present invention relates to a training inserter for an intrauterine system, said intrauterine system comprising a body and removal strings. The invention also relates to a training kit.

### BACKGROUND OF THE INVENTION

Intrauterine systems comprising a body and removal strings are used since some decades. They are typically inserted into the uterus by using an inserter specifically designed for this purpose. In order for medical practitioners to train the insertion of an intrauterine system, regular inserters with intrauterine systems having long removal strings are used. Indeed, the removal strings need to stay inside the inserter even when the intrauterine system itself is released from the inserter, in order to re-introduce the intrauterine system to the inserter for further training. Otherwise, the inserter could only be used for training once.

Re-usable training inserters exist, but the long removal strings are loose at their other end, i.e. the end that is not attached to the intrauterine system. Re-usable training inserters have the advantage of reducing costs for the training and limiting the amount of waste. The long, loose removal strings however create several problems. Indeed, they get easily entangled with the inserter or the fingers of the person using it, and they also give the person a wrong impression of the actual intrauterine system (i.e. on the length of the removal strings). The intrauterine system may also become completely separated from the inserter, if the inserter is not handled with care.

Another problem encountered with the current training inserters is their limited usable life. Indeed, it has been found that since the person needs to pull the intrauterine system back inside the inserter by hand, a too great force is often used, resulting in the breakage of the intrauterine system, the inserter or both or detachment of the removal strings from the intrauterine system. Sometimes a training inserter can only be used two or three times. In addition, the training inserter seems to require more precise user instructions than the inserter used with patients for inserting real intrauterine systems, thus creating a false impression of difficulty of use for the medical practitioner.

### OBJECTS AND SUMMARY OF THE INVENTION

It is an object of the invention to provide a more reliable training inserter, that is easy to use and that can be used several times. Another object of the invention is to provide a training inserter that differs in a distinct and easily recognisable manner from the inserter used for patients.

The invention relates to a training inserter according to the enclosed independent claim. The dependent claims disclose some embodiments of the invention.

A typical training inserter according to the present invention is for an intrauterine system comprising a body and removal strings, and the training inserter comprises
a handle having a first end and a second end,
an insertion tube having a first end and a second end, and being arranged in connection with the handle, for receiving the intrauterine system,
a plunger arranged inside the insertion tube, and
first means for moving the insertion tube with respect to the plunger for pushing out the intrauterine system.

A typical training inserter further comprises second means
for attaching the removal strings to the inserter,
for pulling back the intrauterine system via the removal strings, and
for storing the removal strings.

The invention also relates to a training kit comprising a training inserter according to this invention, and a training intrauterine system.

### BRIEF DESCRIPTION OF THE DRAWING

FIG. 1 shows a training inserter according to a first embodiment with the training intrauterine system in a first position.

FIG. 2 shows a training inserter according to the first embodiment with the training intrauterine system in a second position.

FIG. 3 shows a training inserter according to the first embodiment with the training intrauterine system in a third position.

FIG. 4 shows a training inserter according to a second embodiment.

### DETAILED DESCRIPTION OF THE INVENTION

A typical training inserter according to the present invention is thus for an intrauterine system comprising a body and removal strings, and the training inserter comprises
a handle having a first end and a second end,
an insertion tube having a first end and a second end, and being arranged in connection with the handle, for receiving the intrauterine system,
a plunger arranged inside the insertion tube, and
first means for moving the insertion tube with respect to the plunger for pushing out the intrauterine system.

A typical training inserter further comprises second means
for attaching the removal strings to the inserter,
for pulling back the intrauterine system via the removal strings, and
for storing the removal strings.

The training inserter thus comprises all the parts that are comprised in an inserter for an actual intrauterine system, i.e. one that is used on patients (a “real” inserter). It however also comprises second means that allow to at least mostly overcome the problems encountered with the current training inserters. Indeed, the second means attach the removal strings to the inserter, thus avoiding the complete separation of the intrauterine system from the training inserter. The second means are also used to pull the intrauterine system back inside the insertion tube, so that it can be used again for training. The second means is also designed such that no excessive force can be used for pulling the intrauterine system back, thus avoiding breaking the intrauterine system or the training inserter or detachment of the removal strings from the intrauterine system. Moreover, the second means stores the removal strings so that they are not visible to the user and thus cannot get entangled with the user's fingers, for example.

In a typical training inserter, the longitudinal axis of the handle, of the insertion tube and of the plunger are either the same or parallel to each other. Typically, the first ends are also at the same sides, as well as the second ends. The insertion tube may be attached to the handle either in a fixed manner or in a movable manner, and the first means for moving the insertion tube may be for example a knob or a slider, as will be explained in more detail below. Some embodiments of the inserter as such are explained in more detail below, but also in documents EP 2 352 740, EP 2 349 143, EP 2 352 469 and WO 2012/056105, all of which relevant contents are hereby incorporated by reference.

According to an embodiment, said second means comprises a spool for storing the removal strings, and in that the pulling back of the intrauterine system is performed by rotating the spool, and thereby coiling the removal strings around the spool. The second means may thus comprise a spool around which the removal strings can be arranged. Preferably the second means comprises also a disc attached to the spool, for rotating said spool. The disc may be arranged on the outside of the handle, while the spool is arranged inside the handle. This further enhances the fact that the training inserter has a look that noticeably differs from that of the “real” inserter. The disc may be fixed or removable as desired.

According to another embodiment, said second means comprises a movable stick arranged on the handle and for storing the removal strings, and in that the pulling back of the intrauterine system is performed by pulling the stick outwards from the handle. Such a stick thus comprises means for storing the removal strings, for example the ends of the removal strings are attached to the distal end of the stick (i.e. the end of the stick that is further away from the insertion tube) and preferably arranged to run along the length of the stick in a longitudinal cavity or similar. Such a cavity or similar will then help the removal strings not to get entangled when the stick is moved. In a preferred embodiment, the stick is made of two parts and the removal strings arranged inside the stick. In this embodiment, the length of the stick needs to be such that the pulling of the stick makes the intrauterine system re-enter the inserter. The stick may also be arranged either inside the handle or on its outer surface. When arranged inside the handle, a part of it preferably protrudes from the handle even when the intrauterine system is out of the inserter, in order to make the training inserter visually different from the “real” inserter.

According to yet another embodiment, said second means is removably attached to the handle. The second means may also be fixedly attached to the handle.

As mentioned above, the training inserter may have all the features of an actual inserter. For example, the handle may have a longitudinal opening at its first end, said opening having a longitudinal axis parallel to the longitudinal axis of the inserter, a first end and a second end, and further comprise a movable slider arranged in said longitudinal opening and having a first end and a second end, the slider functioning as the first means. The slider is thus the means for moving the insertion tube with respect to the plunger for pushing out the intrauterine system. According to another embodiment, the second end of the insertion tube is attached to the handle. The training inserter may further comprise a flange arranged on the insertion tube, the flange being used for setting the correct insertion depth.

The training inserter may yet further comprise locking means for reversibly locking the intrauterine system in relation to the plunger via a removal string of the intrauterine system, said locking means being controllable by the first means and/or the insertion tube. A special advantage of the present training inserter compared to the previous training inserter where the removal strings are loose, is that pulling back of the intrauterine system can be prevented if the locking means are locked, thus further avoiding breaking the training inserter or the intrauterine system.

The invention also relates to a training kit comprising a training inserter according to this invention, and a training intrauterine system. By training intrauterine system it is meant a system that has the same size and shape as the actual intrauterine system, but does not contain the medication. Such systems may be provided for example in connection with intrauterine systems such as Mirena®, for the training of the physicians. The kit may further comprise a model simulating a vagina and a uterus for practising the insertion of the intrauterine system.

### DETAILED DESCRIPTION OF THE DRAWING

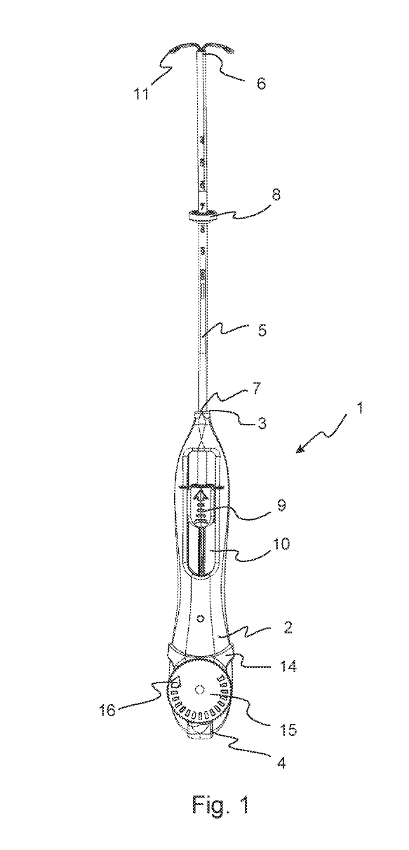

FIG. 1 shows a training inserter according to a first embodiment with the training intrauterine system in a first position. The training inserter 1 comprises a handle 2 having a first end 3 and a second end 4, as well as an insertion tube 5 having a first end 6 and a second end 7, and being arranged in connection with the handle, for receiving the intrauterine system. Indeed, the second end 7 of the insertion tube 5 is attached to the first end 3 of the handle 2. In this embodiment, the insertion tube also comprises an area with graduated markings and a flange 8 used for setting the insertion depth. The inserter also comprises a plunger arranged inside the insertion tube (not shown) and first means for moving the insertion tube with respect to the plunger for pushing out the intrauterine system. In this embodiment, the first means is in the form of a knob 9 arranged into an opening 10 in the handle. The training intrauterine system's body 11 is arranged such that its stem is inside the insertion tube and its T-shaped wings outside the insertion tube. The training intrauterine system's body 11 is thus in the process of being inserted, as the first means (i.e. the knob 9) is half way through the opening 10.

The training inserter 1 further comprises second means 14 arranged on the handle 2 at its second end 4, which second means 14 comprise a spool (inside the handle, not shown) and a disc 15 for turning the spool. The second means 14 thus performs the functions of attaching the removal strings to the inserter, pulling back the intrauterine system via the removal strings by rotating the disc 15, thus the spool, and for storing the removal strings around the spool. The disc also comprises a visual aid in the form of an arrow 16 for showing the direction of rotation of the disc.

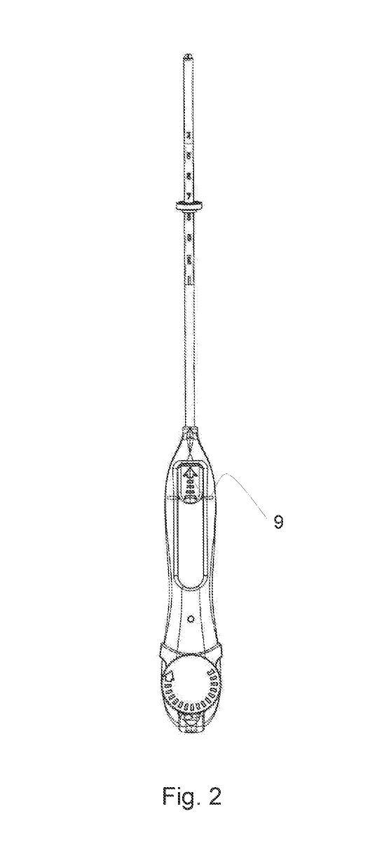

FIG. 2 shows a training inserter according to the first embodiment with the training intrauterine system in a second position, wherein the training intrauterine system is fully inside the training inserter, and the first means (i.e. the knob 9) is at the first end of the handle.

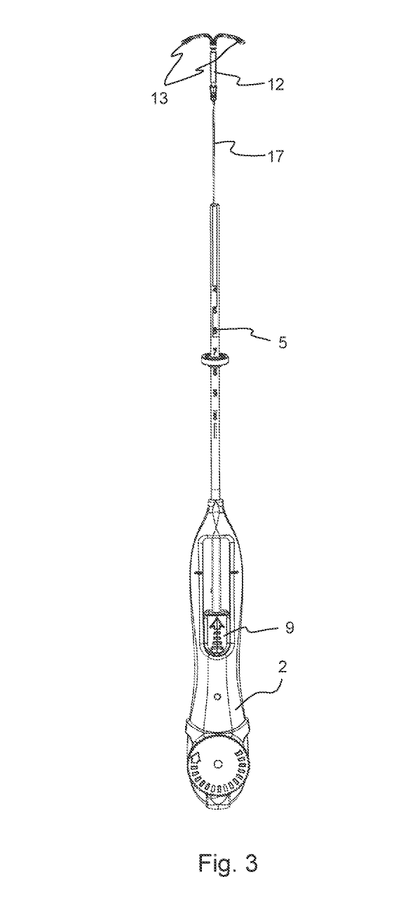

FIG. 3 shows a training inserter according to the first embodiment with the training intrauterine system in a third position. The stem 12 and the T-shaped wings 13 of the training intrauterine system are visible in this Figure, as are the removal strings 17. In this Figure, the training intrauterine system has been inserted, i.e. expulsed from the inserter, and the first means (knob 9) has already been pulled back to its initial position towards the second end of the handle. The person training then only needs to rotate the disc 15 in order for the training intrauterine system's body 11 to re-enter the insertion tube 5. Typically, the training intrauterine system is re-entered to the insertion tube 5 until only the tips of the T-shaped wings 13 are visible.

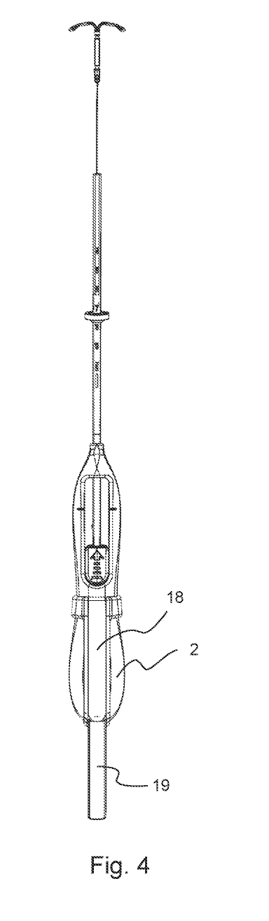

FIG. 4 shows a training inserter according to a second embodiment. In this embodiment, the second means 18 comprises a movable stick 19 arranged on the handle and for storing the removal strings. With this embodiment, the pulling back of the intrauterine system is performed by pulling the stick 19 outwards from the handle 2. In other respects, the training inserter functions as in the first embodiment.

1. A training inserter for an intrauterine system, the intrauterine system comprising a body and removal strings, the training inserter comprising:
a handle having a first end and a second end,
an insertion tube having a first end and a second end, and being arranged in connection with the handle, for receiving the intrauterine system,
a plunger arranged inside the insertion tube, and
a first means for moving the insertion tube with respect to the plunger for pushing out the intrauterine system,
characterized in that it further comprises a second means:
for attaching the removal strings to the inserter,
for pulling back the intrauterine system via the removal strings, and
for storing the removal strings.

2. A training inserter according to claim 1, characterized in that the second means comprises a spool for storing the removal strings, and in that the pulling back of the intrauterine system is performed by rotating the spool.

3. A training inserter according to claim 1, characterized in that the second means comprises a movable stick arranged on the handle and for storing the removal strings, and in that the pulling back of the intrauterine system is performed by pulling the stick outwards from the handle.

4. A training inserter according to claim 1, characterized in that the second means is removably attached to the handle.

5. A training inserter according to claim 1, characterized in that the handle has a longitudinal opening at its first end, the opening having a longitudinal axis parallel to the longitudinal axis of the inserter, a first end and a second end, and further comprising a movable slider arranged in the longitudinal opening and having a first end and a second end, the slider functioning as the first means.

6. A training inserter according to claim 5, characterized in that the second end of the insertion tube is attached to the handle.

7. A training inserter according to claim 1, characterized in that it further comprises a flange arranged on the insertion tube.

8. A training inserter according to claim 1, characterized in that it further comprises locking means for reversibly locking the intrauterine system in relation to the plunger via a removal string of the intrauterine system, the locking means being controllable by the slider and/or the insertion tube.

9. A training kit comprising a training inserter according claim 1 and a training intrauterine system.

10. A training inserter for an intrauterine system, the intrauterine system comprising a body and removal strings, the training inserter comprising:
a handle having a first end and a second end,
an insertion tube having a first end and a second end, and being arranged in connection with the handle, for receiving the intrauterine system,
a plunger arranged inside the insertion tube, and
a slider or knob configured to move the insertion tube with respect to the plunger for pushing out the intrauterine system,
characterized in that it further comprises a spool or a movable stick:
for attaching the removal strings to the inserter,
for pulling back the intrauterine system via the removal strings, and
for storing the removal strings.
